(19) United States
(12) Patent Application Publication
(10) Pub. No.: US 2025/0269076 A1
(43) Pub. Date: Aug. 28, 2025

(54) Apparatus for Mitigating Contamination in Fuel Tanks

(71) Applicant: Vincent Loccisano, Wellesley, MA (US)

(72) Inventor: Vincent Loccisano, Wellesley, MA (US)

(21) Appl. No.: 19/207,692

(22) Filed: May 14, 2025

(51) Int. Cl. A61L 2/025; B01D 17/02; B01F 23/41; B01F 23/411; B01F 31/86; B01F 101/00

(52) U.S. Cl. CPC A61L 2/025; B01D 17/02; B01F 23/4111; B01F 23/4145; B01F 31/86; A61L 2202/11; B01F 2101/505

(57) ABSTRACT

An apparatus for mitigating contamination in a fuel supply is an ultrasonic generator coupled with a piezoelectric transducer that propagates ultrasonic waves through a fuel tank. In some embodiments the apparatus is used to mitigate the growth of microbial organisms. In other embodiments the apparatus is used to emulsify water into the fuel, particularly in diesel fuel. The apparatus addresses water and microbial growth found in boats, cars, and aircraft as well as in storage tanks such as those used at fuel stations. In some embodiments, the fluid is diesel fuel.

### TECHNICAL FIELD

The present disclosure relates generally to methods and apparatuses for controlling contamination in fuel, and more specifically to ultrasonic wave generators for controlling microbial growth and water contamination in diesel fuel.

### BACKGROUND OF THE INVENTION

Microbial contamination of diesel fuel is a persistent and costly problem affecting storage, transportation and use of this critical energy source. The proliferation of bacteria, fungi, and yeasts (often inaccurately described as “algae”) in fuel tanks, particularly in the nutrient-rich environment of modern Ultra-Low Sulfur Diesel (ULSD) and biodiesel blends, leads to a cascade of detrimental effects. These include the formation of obstructive biofilms and biomass: fuel degradation: corrosion of fuel-system components; inaccurate fuel-level readings; reduced engine performance; and potential system failure.

Diesel fuel, when contaminated with water, diesel fuel can become a breeding ground for various microbial contaminants. These microorganisms live in the water phase and feed on the hydrocarbons in the diesel fuel at the fuel/water interface. The primary types of microbial contaminants found in diesel fuel include bacteria including aerobic and anaerobic bacteria, usually found in the form of sludge at the bottom of a storage tank. Sulfate-reducing bacteria are a type of anerobic bacteria that produce hydrogen sulfide which is corrosive to steel tanks and components. Other common bacteria include Pseudomonas, known for forming biofilms, Acinetobacter, Bacillus and species from the enterobacteriaceae family, Hormoconis resinae, Fusarium and Botrytis. Fungi in the form of molds and yeasts include Cladosporium resinae, often referred to as kerosene fungus or diesel bug, Aspergilus and penicillin may also be found. Other yeasts include Candida, Yarrowia, Rhodotorula, Torula, Saccharomyces and Hansenula.

For the general disinfection of diesel fuel from a mix of microbial contaminants, including bacteria like Pseudomonas, Acinetobacter, Bacillus, and fungi like Hormoconis resinae, Fusarium, and Botrytis, ultrasonic frequencies in the range of 20 kHz to a little over 40 kHz (e.g., 42 kHz) are most commonly reported or implied in the context of fuel treatment.

Existing strategies for mitigating microbial growth in diesel fuel encompass chemical biocides, physical removal through filtration and fuel polishing, and preventative measures such as water management. While biocides can offer immediate control, they pose environmental and handling concerns; can be ineffective against established biofilms; and may contribute to the development of resistant microbial strains. Physical removal methods, while essential for eliminating existing particulate matter, do not prevent microbial regrowth.

Ultrasonic cavitation involves generating microscopic vacuum bubbles in a liquid medium and subjecting the liquid to high-intensity sound waves. The subsequent violent collapse of these cavitation bubbles creates intensely localized and transient extreme conditions—including high temperatures, high pressures, and high-velocity micro-jets—capable of physically damaging outer membranes of microbial cells and disrupting the structural integrity of biofilms. These high-velocity micro-jets are also capable of breaking up water droplets to emulsify water into diesel fuel.

Ultrasonic emulsification of water in diesel relies on the intense acoustic cavitation generated by ultrasonic waves. The rapid formation and violent collapse of cavitation bubbles create high shear forces and turbulence. These forces break down the interfacial tension between water and diesel phases, shattering larger droplets into smaller droplets, often in the micron or nano-size range. The rapid evaporation of the finely dispersed water droplets within the combustion chamber leads to a phenomenon called micro-explosion. This secondary atomization further breaks down the fuel droplets resulting in more complete combustion of diesel fuel.

Piezoelectric materials convert mechanical energy into electrical energy, and vice versa, in a phenomenon known as the piezoelectric effect. An alternating electrical voltage applied across a piezoelectric element causes it to mechanically vibrate at a frequency corresponding to the voltage. When operated at ultrasonic frequencies (above 20 kHz), these vibrations generate high-frequency sound waves that propagate through a surrounding medium, inducing an ultrasonic cavitation effect.

### SUMMARY OF THE INVENTION

An apparatus for mitigating contamination in a fuel supply is an ultrasonic generator coupled with a piezoelectric transducer that propagates ultrasonic waves through a fuel tank. In some embodiments the apparatus is used to mitigate the growth of microbial organisms. In other embodiments the apparatus is used to emulsify water into the fuel, particularly in diesel fuel. The apparatus addresses water and microbial growth found in boats, cars, and aircraft as well as in storage tanks such as those used at fuel stations. In some embodiments, the fluid is diesel fuel.

In an example embodiment an ultrasonic generator coupled with a piezoelectric transducer is affixed to the wall of a storage tank or an on-board fuel tank in a vehicle. In some embodiments the ultrasonic generator and piezoelectric transducer combination is in contact with the fluid fuel, while in other embodiments the apparatus is affixed to the wall of the tank. One skilled in the art understands that ultrasonic waves may vibrate a tank wall and further vibrate through a fluid as well as propagate directly through a fluid.

In another embodiment, the generator/transducer combination is housed in a fluid-tight, immersible housing, which may be pre-installed or after-market-installed onto a fuel-tank wall or internal baffle. In an embodiment, the apparatus is installed inside the tank at time of manufacture. In another embodiment, the apparatus is installed post-manufacturing. The apparatus may be bonded to the tank's exterior at the time of manufacture or post-manufacturing. Yet another embodiment has the apparatus affixed, in its housing, to a flotation device that suspends the piezoelectric transducer on the surface of a fluid. In yet another embodiment the apparatus is affixed to a housing designed to be temporarily affixed to a tank for intermittent mitigation of water and microbial growth in the tank.

In yet another embodiment, the apparatus is affixed to a structural component of a fuel-level sensor. The apparatus is affixed to an arm that supports a float for determining the fuel level. In this configuration, the apparatus is suspended near the surface of the fuel and remains at the surface as the fuel level rises and falls. As the fuel level fluctuates, the piezoelectric transducer changes angle, propagating ultrasonic waves through the fuel at varying angles so as to reach all areas of the fuel tank.

The specific ultrasound frequency may be in the range of 35 kHz to 40 kHz to be effective for various bacterial species. Higher frequencies, up to 150 kHz, may also be produced.

### BRIEF DESCRIPTION OF DRAWINGS

FIG. 1 is an illustration of the embodiment.

FIG. 2A is a detailed, exploded view illustrating the embodiment in a housing, mounted to a tank with direct contact with fluid fuel in the tank.

FIG. 2B is a detailed, exploded view illustrating the embodiment in a housing, mounted to a tank surface.

FIG. 2C is a detail view of FIG. 2A or FIG. 2B.

FIG. 3 is an illustration of an iteration of the embodiment, mounted on a fuel filter apparatus.

FIG. 4 is an illustration of an iteration of the embodiment.

FIG. 5 is an illustration thereof mounted on a portion of a fuel level sensor.

FIG. 6 is an illustration thereof, showing the embodiment's and a float valve's movement.

### DETAILED DESCRIPTION

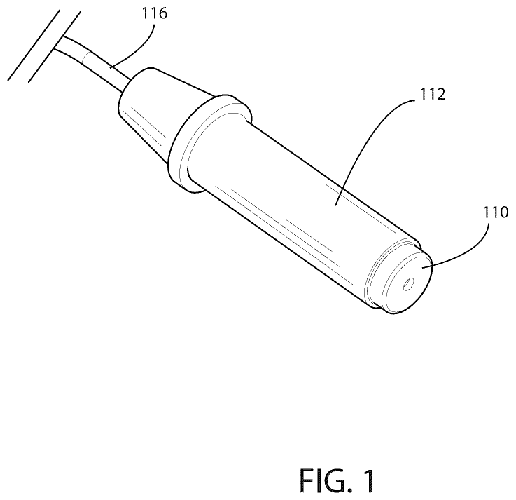

In FIG. 1, an example embodiment is a piezoelectric transducer 110 coupled with an ultrasonic generator 112. Wires 116 provide 12V power to the apparatus.

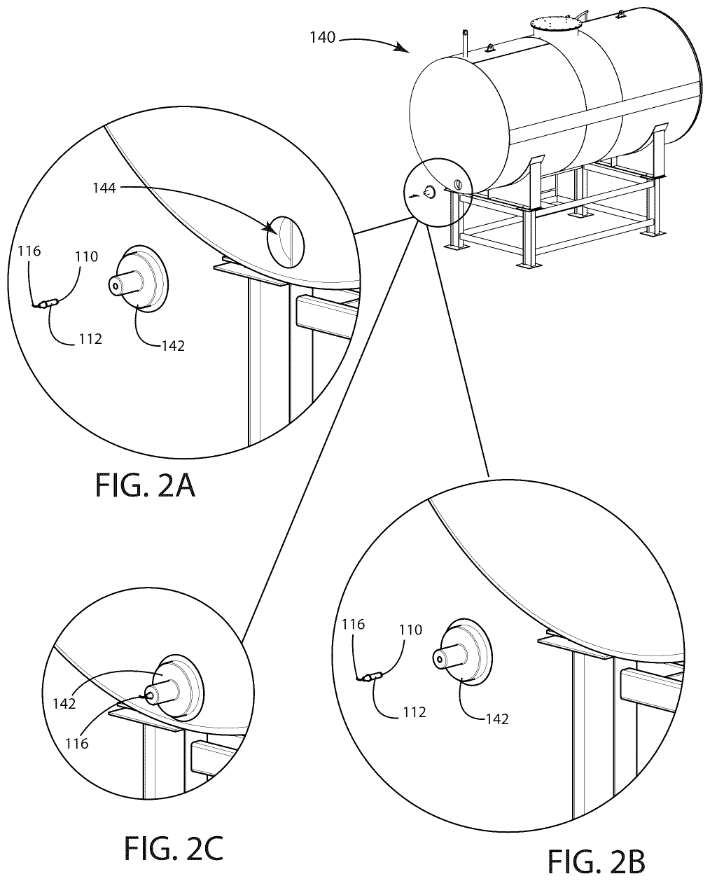

FIG. 2A is a detail, exploded view of the apparatus of FIG. 1 installed in a sidewall of a fuel storage tank. The piezoelectric transducer 110 coupled with an ultrasonic generator 112 is sealed in a flange 142 that is affixed against a surface of a fuel storage tank 140 and sealed around an opening 144 in the tank wall. Wires 116 extend out of the flange 142. In this example embodiment, the piezoelectric transducer 110 is in contact with the fluid fuel in the tank.

FIG. 2B is a detail, exploded view of the apparatus of FIG. 1 installed against the exterior of a sidewall of a fuel storage tank. The piezoelectric transducer 110 and ultrasonic generator 112 is sealed in a flange 142 that is affixed against a surface of a fuel storage tank 140 and sealed against a wall of the tank 140. Wires 116 extend out of the flange 142. In this example embodiment, the piezoelectric transducer 110 is in contact with the outer surface of the tank 140. Ultrasonic waves are propagated through the tank wall and propagate through the wall and fluid fuel in the tank. FIG. 2C illustrates either the embodiment of FIG. 2A or FIG. 2B as installed. One skilled in the art understands that the embodiment in FIG. 2B may be installed temporarily on a tank to provide microbial growth mitigation on any number of tanks.

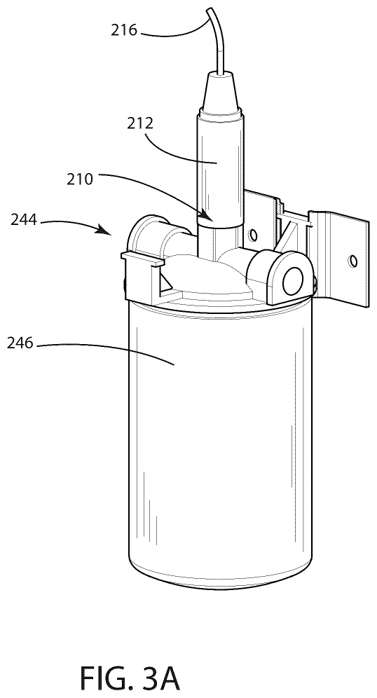

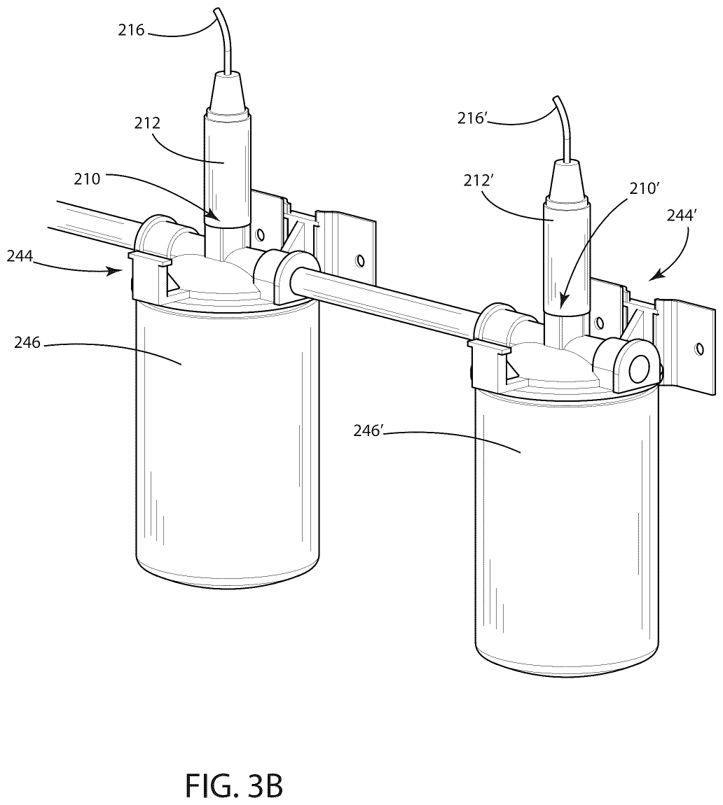

FIG. 3A illustrates an iteration of the embodiment engaged with a fuel filter fixture 244. A fuel filter 246 is affixed to the fixture 244. A piezoelectric transducer 210 (110 in FIG. 1) coupled with an ultrasonic generator 212 and affixed to an outer surface of the fixture 244. Power is supplied to the piezoelectric transducer 210 through power cable 216. Ultrasonic waves propagate through fuel as it passes through the fixture 244 and filter 246 to separate blended or emulsified contaminants to improve the efficacy of the filter 246.

FIG. 3B illustrates an iteration of the embodiment engaged with a series of fuel filter fixtures 244, 244′ . . . . In the example embodiment, a fuel filter 246 is affixed to the fixture 244, while a fuel filter 246′ is affixed to fixture 244′. A first piezoelectric transducer 210 is coupled with an ultrasonic generator 212 and affixed to an outer surface of the fixture 244. A second piezoelectric transducer 210′ is coupled with an ultrasonic generator 212′ and affixed to an outer surface of the fixture 244′. Power is supplied to the piezoelectric transducer 210 through power cable 216, and to the piezoelectric transducer 210′ through power cable 216′. Ultrasonic waves propagate through fuel as it passes through the fixture 244, filter 246, fixture 244′ and filter 246′ to provide a staged process to separate blended or emulsified contaminants from the fuel. A first stage of separation occurs at a first frequency in fixture 244. A second stage of separation occurs at a second frequency in fixture 244′. One skilled in the art understands that a series of any number of fixtures, filters and piezoelectric transducers, producing various frequencies for various contaminants may be configured.

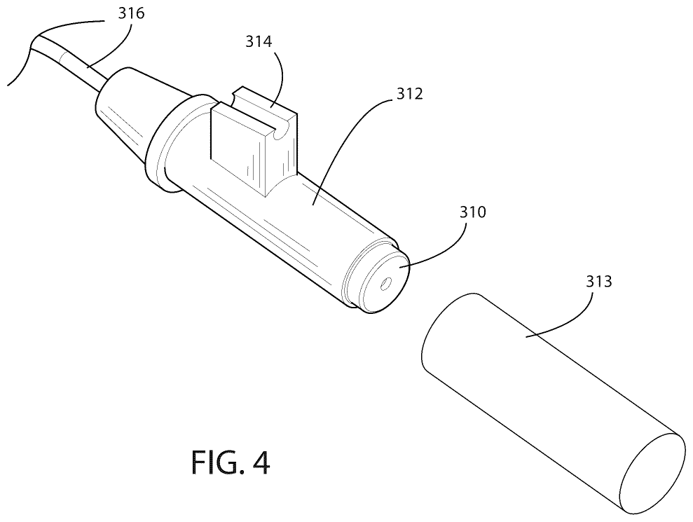

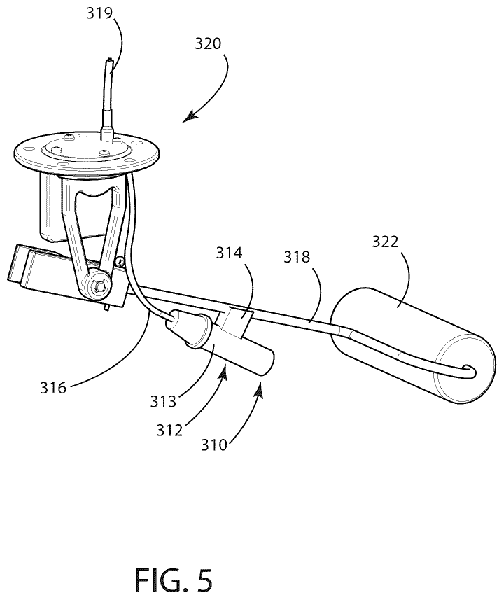

FIG. 4 shows an example embodiment having a piezoelectric transducer 310 coupled with an ultrasonic generator 312 is sealed in a fluid-tight housing 313. Wires 316 are joined to a 12V vehicle power system to provide 12V power to the piezoelectric transducer 310. The immersible housing 313 has a mounting mechanism 314 configured to mount the immersible transducer/ultrasonic generator 310/312 on an arm of a fuel level sensor (FIG. 5).

FIG. 5 shows the apparatus of FIG. 4 mounted to a structural element of a fuel-level sensor 320. In some embodiments, the mounting mechanism 314 is configured to affix to an arm 318 on a fuel-level sensor 320 which is affixed to a float 322. The piezoelectric transducer 310 and ultrasonic generator 312 in the immersible housing 313 is powered by a power cable 316 that is electrically coupled with a power cable 319 for the fuel-level sensor 320. In some embodiments the power source is a 12V automobile power source.

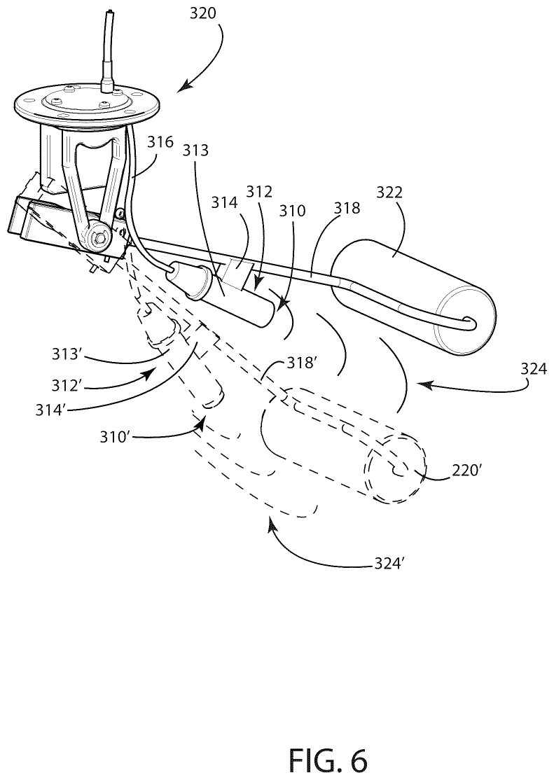

FIG. 6 illustrates the apparatus in motion. The apparatus 310 and 310′ and ultrasonic generator 312 and 312′ and immersible housing 313 are shown with the mounting mechanism 314 and 314′ affixed to an arm 318 and 318′ that is in turn affixed to a float 320 and 320′ on a fuel-level sensor 320. Ultrasonic waves 324 and 324′ are propagated through fuel upon which the float 322, 322′ rests. The ultrasonic waves 324 and 324′ are propagated at differing angles through a body of fuel as the fuel level goes up and down, as demonstrated by ultrasonic waves 324′ propagated at a different angle than differing ultrasonic waves 324.

The apparatus has been shown here affixed to a fuel-level sensor 320 common in internal-combustion vehicles. One skilled in the art understands that the apparatus may be affixed to the fuel-level sensor of any fuel tank.

1. An apparatus for mitigating contamination in a fuel supply comprising:
an ultrasonic generator capable of producing at least one frequency coupled with a piezoelectric transducer that is configured to generate ultrasonic waves; and
the ultrasonic waves propagate through a fluid fuel; wherein
the at least one frequency is selected to provide mitigation of contaminates in the fluid fuel.

2. The apparatus of claim 1 wherein:
the at least one frequency is a fixed frequency.

3. The apparatus of claim 1 further comprising:
the at least one frequency is swept through a range of frequencies.

4. The apparatus of claim 1 wherein:
the piezoelectric transducer is fixedly engaged with a fuel tank.

5. The apparatus of claim 1 wherein:
the piezoelectric transducer is fixedly engaged with a fuel level sensor.

6. The apparatus of claim 4 wherein:
the ultrasonic generator coupled with the piezoelectric transducer is fixedly engaged with a flange that is welded to the fuel tank at the time of construction.

7. The apparatus of claim 4 wherein:
ultrasonic generator coupled with the piezoelectric transducer is fixedly engaged with a flange that is sealed to the fuel tank after construction.

8. The apparatus of claim 1 wherein:
ultrasonic generator coupled with the piezoelectric transducer is fixedly engaged with a fluid supply line; wherein
contaminates are separated from fuel along a supply line.

9. The apparatus of claim 8 further comprising:
an array of ultrasonic generators coupled with the piezoelectric transducers are spaced along a fluid supply line; wherein
each ultrasonic generator generates a different wavelength to mitigate different contaminants in the fuel in the supply line.

10. The apparatus of claim 1 wherein:
the ultrasonic generator coupled with the piezoelectric transducer generates a frequency of ultrasound that is between 20 kHz and 100 kHz.

11. The apparatus of claim 1 wherein:
the ultrasonic generator coupled with the piezoelectric transducer generates a frequency of ultrasound that is between 20 kHz and 40 KHz.

12. The apparatus of claim 11 wherein contaminants removed are selected from the group consisting of:
Pseudomonas, Acinetobacter, Bacillus, Hormoconis resinae, Fusarium and Botrytis.

13. The apparatus of claim 11 wherein contaminants removed are selected from the group consisting of:
Cladosporium resinae, Aspergilus, penicillin, Candida, Yarrowia, Rhodotorula, Torula, Saccharomyces and Hansenula.

14. The apparatus of claim 11 wherein water is agglomerated for faster separation.

15. The apparatus of claim 11 wherein asphaltene aggregates are broken to prevent deposition.

16. An apparatus for emulsifying water in a fuel supply comprising:
an ultrasonic generator capable of producing at least one frequency coupled with a piezoelectric transducer that is configured to generate ultrasonic waves; and
the ultrasonic waves propagate through a fluid fuel; wherein the at least one frequency is selected to provide emulsification of water in the fluid fuel.

17. The apparatus of claim 16 wherein:
the ultrasonic generator coupled with the piezoelectric transducer generates a frequency of ultrasound that is between 16 kHz and 50 kHz.

18. The apparatus of claim 16 wherein:
the ultrasonic generator coupled with the piezoelectric transducer generates a frequency of ultrasound that is 28 kHz.

19. The apparatus of claim 16 wherein:
The apparatus is engaged with a fuel level sensor in a fuel tank.

20. The apparatus of claim 16 wherein:
the apparatus is engaged with a fuel supply line.
